SEQUENCE LISTING (8 sequences)

SEQ ID NO: 1 - protein, 8 aa, Artificial
  Synthetic ET1F08 CDR1
GLTISNYA

SEQ ID NO: 2 - protein, 7 aa, Artificial
  Synthetic ET1F08  CDR2
INWSGNK

SEQ ID NO: 3 - protein, 20 aa, Artificial
  Synthetic ET1F08 CDR3
AARFHSYAASTYYSASTYKF

SEQ ID NO: 4 - protein, 93 aa, Artificial
  Synthetic ET1F08
MAQVQLVQSGGGLVQAGGSLRLSCAASMAWFRQAPGKEREFVALYYADSVKGRFTIARDNAKNTVDLQMNSLKPEDTAVYYCWGQGTQVTVSS

SEQ ID NO: 5 - protein, 8 aa, Artificial
  Synthetic ET2F09 CDR1
GFTFDDYA

SEQ ID NO: 6 - protein, 8 aa, Artificial
  Synthetic ET2F09 CDR2
ISWSGRTT

SEQ ID NO: 7 - protein, 13 aa, Artificial
  Synthetic ET2F09 CDR3
ARGDVAIRGNLDA

SEQ ID NO: 8 - protein, 93 aa, Artificial
  Synthetic ET2F09
MAQVQLVQSGGGLVQPGGSLRLSCAASMSWVRQAPGKGLEWVSAYYAESMKGRFTTSRDNAKNTLYLQMNSLKPEDTALYYCWGQGTQVTVSS

(19) United States
(12) Patent Application Publication
(10) Pub. No.: US 2018/0327499 A1
(43) Pub. Date: Nov. 15, 2018

(54) ANTI- NKG2D SINGLE DOMAIN ANTIBODIES AND USES THEREOF

(71) Applicants: INSERM (INSTITUT NATIONAL DE LA SANTÉ ET DE LA RECHERCHE MÉDICALE), Paris (FR); UNIVERSITÉ D'AIX MARSEILLE, Marseille Cedex 07 (FR); INSTITUT JEAN PAOLI & IRENE CALMETTES, Marseille (FR); CENTRE NATIONAL DE LA RECHERCHE SCIENTIFIQUE (CNRS), Paris (FR)

(72) Inventors: Daniel BATY, Marseille Cedex 09 (FR); Patrick CHAMES, Marseille Cedex 09 (FR); Brigitte KERFELEC, Marseille Cedex 09 (FR); Elise TERMINE, Marseille Cedex 09 (FR)

(73) Assignees: INSERM (INSTITUT NATIONAL DE LA SANTÉ ET DE LA RECHERCHE MÉDICALE), Paris (FR); UNIVERSITÉ D'AIX MARSEILLE, Marseille Cedex 07 (FR); INSTITUT JEAN PAOLI & IRENE CALMETTES, Marseille (FR); CENTRE NATIONAL DE LA RECHERCHE SCIENTIFIQUE (CNRS (CNRS), Paris (FR)

(21) Appl. No.: 15/775,579

(22) PCT Filed: Nov. 10, 2016
(86) PCT No.: PCT/EP2016/077326

§ 371 Date: May 11, 2018

(30) Foreign Application Priority Data
Nov. 13, 2015 (EP) 15306809.3

(51) Int. Cl. C07K 16/28; C07K 16/32

(52) U.S. Cl. CPC C07K 16/2851; C07K 16/32; C07K 16/2809; C07K 2317/31; C07K 2317/92; C07K 2317/569; C07K 2317/22; C07K 2317/732; C07K 2317/55

(57) ABSTRACT

The present invention relates to anti-Natural Killer Group 2 member D (NKG2D) single domain antibodies and uses thereof in particular in the therapeutic field.

### FIELD OF THE INVENTION

The present invention relates to anti-Natural Killer Group 2 member D (NKG2D) single domain antibodies and uses thereof in particular in the therapeutic field.

### BACKGROUND OF THE INVENTION

Natural Killer (NK) cells are innate immune cells that control microbial infections and tumors. NK cells express a large number of different cell surface receptors that deliver either activating or inhibitory signals. The function of natural killer cells is regulated by a balance between signals transmitted by activating receptors and inhibitory receptors¹. Inhibitory NK cell receptor recognize MHC class I (CMH I) which is present on normal cells and NK cells do not kill normal cells. Inhibitory receptor NK cell including the human killer cell immunoglobulin-like receptors (KIRs) and the rodent lymphocyte antigen 49 complex (Ly49) receptors²⁻⁵. These receptors contain immunoreceptor tyrosine-based inhibitory motifs (ITIMs) in their cytoplasmic domains, which recruit intracellular tyrosine phosphatases that mediate the inhibition. Conversely, infected cells or tumor cells express low CMH I which allows the activation of NK cells to kill infected cells or tumor cells: the activation of NK cells results from the concerted action of cytokine receptors, adhesion molecules, and interactions between activating receptors recognizing ligands on the surface of tumors or pathogen-infected cells. Several activating receptors have been implicated in the recognition of tumours and virus-infected cells²⁻¹⁰. Most of the activating NK cell receptors are transmembrane molecules with short intracellular domains that lack intrinsic signalling activity. They function by coupling to signal transducing transmembrane adaptor molecules through charged amino acids in their transmembrane regions²⁻¹⁰. The Natural Killer Group 2 member D (NKG2D) is an activating receptor and expressed on killer cells of the innate immune system, including NK cells, NATURAL KILLER T (NKT) cells, γδTCR+ T cells, and also on cells of the acquired immune system, such as CD8+ T cells¹¹. NKG2D is a disulphide-linked type II transmembrane proteins with short intracellular domains incapable of transducing signals. It requires adaptor proteins in order to transduce signals, and this receptor uses two adapter proteins, DAP10 and DAP12, which associate as homodimers to the receptor. NKG2D is encoded by a gene in the NK complex on mouse chromosome 6 and on human chromosome 12. The DAP10 adaptor molecule forms a complex with NKG2D. The non-covalent interaction between NKG2D and DAP10 is required for the cell-surface expression of the functional receptor complex¹³. The DAP10 cytoplasmic domain has a YINM motif and recruits phosphatidylinositol 3-kinase (PI3K)¹⁴⁻¹⁵. In T cells, co-stimulatory molecules, such as CD28 and inducible co-stimulatory molecule (ICOS), use the PI3K signalling pathway, indicating a role for NKG2D as a co-stimulatory molecule on CD8+ T cells. The NKG2D receptor recognizes several MHC class-I-like ligands that show diverse expression patterns and modes of induction. In humans, the highly polymorphic MHC-class-I-chain related A and B antigens (MICA and MICB), encoded by genes within the human MHC, bind to NKG2D. MIC proteins consist of an α1-α2- and α3-domain and do not bind to β2-microglobulin or peptide¹⁶. NKG2D ligands are not expressed on the surface of healthy cells and tissues in adults¹⁷. Every type of cancer is capable of expressing one or more of the NKG2D ligands¹⁷. NKG2D alone is insufficient to trigger cell-mediated cytotoxicity or cytokine production. Simultaneous engagement of NKG2D and other “costimulatory” receptors, such as CD335 (NKp46) or CD244 (2B4) can trigger cytolytic activity in resting human NK cells. Once “primed” by culture in IL2 or IL15, engagement of NKG2D alone is sufficient to initiate degranulation and cytokine production by human NK cells. NKG2D fails to costimulate TCR-induced activation of resting CD8 T cells, and only augments TCR dependent activation after the T cells have been activated and cultured for a period of time in vitro. After some period of culture, human CD8 T cells acquire the capacity to kill ligand-bearing target cells in an NKG2D-dependent, TCR independent fashion. In vitro, when NK cells or T cells are cocultured with NKG2D ligand-bearing cells, NKG2D is downregulated presumably by clusterization and cross-linking of the NKG2D receptors, triggering their internalization in the NK cells and T cells by membrane-bound form of ligands. Thus, it is needed to find a strategy to enhance NKG2D to trigger cell-mediated cytotoxicity or cytokine production.

Recently, the use of non-conventional antibodies has emerged as a simple, new and sensitive approach to study protein conformation on living cells. Single domain antibodies (sdAbs, also called nanobodies)¹⁸ correspond to the variable domains of a special class of antibodies naturally devoid of light chains found in Camelids. These small proteins (13 kDa) present several advantages¹⁹ including a good thermal stability even without disulfide bond formation²⁰, a good solubility and high expression yield²¹. Furthermore, they are well suited for construction of larger molecules and selection systems such as phage, yeast, or ribosome display. More interestingly, sdAbs have a natural tendency to bind epitopes that are inaccessible to conventional antibodies²², such as cleft and cavities. Consequently, they are often very sensitive to conformational changes of their target²³⁻²⁴.

### SUMMARY OF THE INVENTION

The present invention relates to anti-Natural Killer Group 2 member D (NKG2D) single domain antibodies and uses thereof in particular in the therapeutic and prognostic fields. In particular, the present invention is defined by the claims.

### DETAILED DESCRIPTION OF THE INVENTION

The NKG2D is an activating receptor and expressed on killer cells of the innate immune system, including NK cells, NATURAL KILLER T (NKT) cells, γδTCR+ T cells, and also on cells of the acquired immune system, such as CD8+ T cells¹¹. NKG2D is a type II disulphide-linked dimer with a lectin-like extracellular domain¹². It is encoded by a gene in the NK complex on mouse chromosome 6 and on human chromosome 12¹². In the present invention, the inventors describe the selection and characterization of anti-NKG2D single domains antibodies. In particular, the inventors isolated two different specific anti-NKG2D clones. Structure and sequences of said antibodies are depicted in Table A. Interesting, the binding of two single domains antibodies (sdAbs) was found highly sensitive to ligand stimulation. The two Abs, ET1F8 and ET2F9, can only bind the ligand-free NKG2D conformation. ET1F8 is an antigen binding variable domain of the H chain of heavy-chain antibody (VHH) and ET1F9 is a variable domain of the heavy chain of immunoglobulins (VH).

The two anti-NKG2D single domains antibodies isolated have high affinity to bind to NKG2D and to stimulate cytotoxicity by NK cells.

### Anti-NKG2D Single Domain Antibodies

Accordingly, the invention relates to an isolated single domain antibody directed against natural killer group 2 member D (NKG2D) comprising:
a CDR1 having least 70% of identity with sequence set forth as SEQ ID NO:1,
a CDR2 having at least 70% of identity with sequence set forth as SEQ ID NO:2,
a CDR3 having at least 70% of identity with sequence set forth as SEQ ID NO:3;
   wherein said isolated single domain antibody has the sequence set forth as SEQ ID NO:4 or
a CDR1 having least 70% of identity with sequence set forth as SEQ ID NO:5,
a CDR2 having at least 70% of identity with sequence set forth as SEQ ID NO:6,
CDR3 having at least 70% of identity with sequence set forth as SEQ ID NO:7,
   wherein, said isolated single domain antibody has the sequence set forth as SEQ ID NO: 8.

These antibodies can be very useful for designing new therapeutic and prognostic tools.

TABLE A

| | SEQ | |
|---|---|---|
| Name | ID NO: | Sequence |
| ET1F08 | 1 | GLTISNYA |
| CDR1 | | |
| ET1F08 | 2 | INWSGNK |
| CDR2 | | |
| ET1F08 | 3 | AARFHSYAASTYYSASTYKF |
| CDR3 | | |
| ET1F08 | 4 | MAQVQ LVQSGG GLVQAGGSLRLSCAAS |
| | | MAWFRQAPGKEREFVAL YYADSVK |
| | | GRFTIARDNAKNTVDLQMNSLKPEDTAVYYC |
| | | WGQGTQ V TVSS |
| ET2F09 | 5 | GFTF DDYA |
| CDR1 | | |
| ET2F09 | 6 | ISWS GRTT |
| CDR2 | | |
| ET2F09 | 7 | ARGDVAI RGNLDA |
| CDR3 | | |
| ET2F09 | 8 | MAQVQ LVQSGG GLVQPGGSLRLSCAAS |
| | | MSWVRQAPGKGLEWVSA YYAESMK |
| | | GRFTTSRDNAKNTLYLQMNSLKPEDTALYYC |
| | | WGQGTQ V TVSS |

As used herein the term “single domain antibody” has its general meaning in the art and refers to the single heavy chain variable domain of antibodies of the type that can be found in Camelid mammals which are naturally devoid of light chains. Such single domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 1” or “CDR2” and as “Complementarity Determining Region 2” or “CDR3” and as “Complementarity Determining Region 2”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH domains given by the International ImMunoGeneTics information system aminoacid numbering (http://imgt.cines.fr/).

In particular, the present invention relates to an isolated single domain antibody (ET1F8 derivate) comprising a CDR1 having at least 70% of identity with sequence set forth as SEQ ID NO:1, a CDR2 having at least 70% of identity with sequence set forth as SEQ ID NO:2 and a CDR3 having at least 70% of identity with sequence set forth as SEQ ID NO:3.

In particular, the present invention relates to an isolated single domain antibody (ET2F09 derivate) comprising a CDR1 having at least 70% of identity with sequence set forth as SEQ ID NO:5, a CDR2 having at least 70% of identity with sequence set forth as SEQ ID NO:6 and a CDR3 having at least 70% of identity with sequence set forth as SEQ ID NO:7.

According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with the second amino acid sequence. Amino acid sequence identity is typically determined using a suitable sequence alignment algorithm and default parameters, such as BLAST P (Karlin and Altschul, 1990).

In some embodiments the isolated single domain antibody (ET1F8) according to the invention comprises a CDR1 having a sequence set forth as SEQ ID NO: 1, a CDR2 having a sequence set forth as SEQ ID NO:2 and a CDR3 having a sequence set forth as SEQ ID NO:3.

In some embodiments the isolated single domain antibody (ET2F09) according to the invention comprises a CDR1 having a sequence set forth as SEQ ID NO:5, a CDR2 having a sequence set forth as SEQ ID NO:6 and a CDR3 having a sequence set forth as SEQ ID NO:7.

In some embodiments, the isolated single domain antibody according to the invention has the sequence set forth as SEQ ID NO:4 (“ET1F8”).

In some embodiments, the isolated single domain antibody according to the invention has the sequence set forth as SEQ ID NO:8 (“ET2F09”).

In some embodiments, the single domain antibody is a “humanized” single domain antibody. As used herein the term “humanized” refers to a single domain antibody of the invention wherein an amino acid sequence that corresponds to the amino acid sequence of a naturally occurring VHH domain has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional chain antibody from a human being. Methods for humanizing single domain antibodies are well known in the art. Typically, the humanizing substitutions should be chosen such that the resulting humanized single domain antibodies still retain the favorable properties of single domain antibodies of the invention. The one skilled in the art is able to determine and select suitable humanizing substitutions or suitable combinations of humanizing substitutions. For example, the single domain antibodies of the invention may be suitably humanized at any framework residue depicted in Figure S1 provided that the single domain antibodies remain soluble and do not significantly loss their affinity for NKG2D.

A further aspect of the invention refers to a polypeptide comprising at least one single domain antibody of the invention.

Typically, the polypeptide of the invention comprises a single domain antibody of the invention, which is fused at its N terminal end, at its C terminal end, or both at its N terminal end and at its C terminal end to at least one further amino acid sequence, i.e. so as to provide a fusion protein. According to the invention the polypeptides that comprise a sole single domain antibody are referred to herein as “monovalent” polypeptides. Polypeptides that comprise or essentially consist of two or more single domain antibodies according to the invention are referred to herein as “multivalent” polypeptides.

In some embodiments, the polypeptide comprises at least one single domain antibody of the invention and at least one other binding unit directed against another epitope, antigen, target, protein or polypeptide of tumour cells, which is typically also a single domain antibody. Such a polypeptide is referred to herein as “multispecific” polypeptide; in opposition to a polypeptide comprising the same single domain antibodies (“monospecific” polypeptide). Thus, in some embodiments, the polypeptide of the invention may also provide at least one further binding site directed against any desired protein, polypeptide, antigen, antigenic determinant or epitope. Said binding site is directed against to the same protein, polypeptide, antigen, antigenic determinant or epitope for which the single domain antibody of the invention is directed against, or may be directed against a different protein, polypeptide, antigen, antigenic determinant or epitope) from the single domain antibody of the invention.

A “bispecific” polypeptide of the invention is a polypeptide that comprises at least one single domain antibody (ET1F08 or ET2F09) directed against a first antigen (i.e. NKG2D) and at least one further binding site directed against a second antigen (i.e. different from NKG2D), whereas a “trispecific” polypeptide of the invention is a polypeptide that comprises at least one single domain antibody directed against a first antigen (i.e. NKG2D), at least one further binding site directed against a second antigen (i.e. different from NKG2D) and at least one further binding site directed against a third antigen (i.e. different from both i.e. first and second antigen); etc.

In some embodiments, the first binding site is directed against NKG2D and the further binding site directed against a second antigen. Typically, the further binding site may be directed against a cancer antigen. Cancer antigen refers to all protein, peptide, polypeptide or fragments present on the tumors cells. Known cancer antigens include, without limitation, c-erbB-2 (erbB-2 is also known as c-neu or HER-2), which is particularly associated with breast, ovarian, and colon tumor cells, as well as neuroblastoma, lung cancer, thyroid cancer, pancreatic cancer, prostate cancer, renal cancer and cancers of the digestive tract. Another class of cancer antigens is oncofetal proteins of nonenzymatic function. These antigens are found in a variety of neoplasms, and are often referred to as “tumor-associated antigens.” Carcinoembryonic antigen (CEA), and α-fetoprotein (AFP) are two examples of such cancer antigens. AFP levels rise in patients with hepatocellular carcinoma: 69% of patients with liver cancer express high levels of AFP in their serum. CEA is a serum glycoprotein of 200 kDa found in adenocarcinoma of colon, as well as cancers of the lung and genitourinary tract. Yet another class of cancer antigens is those antigens unique to a particular tumor, referred to sometimes as “tumor specific antigens,” such as heat shock proteins (e.g., hsp70 or hsp90 proteins) from a particular type of tumor.

Additional specific examples of cancer antigens include epithelial cell adhesion molecule (Ep-CAM/TACSTD1), mesothelin, tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA, PSMA), RAGE (renal antigen), CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Mart1, MelanA, MUM-1, MUM-2, MUM-3, HLA-B7, Ep-CAM, tumor-derived heat shock proteins, and the like (see also, e.g., Acres et al., Curr Opin Mol Ther 2004 February, 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). Other exemplary cancer antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell cancer antigens described in U.S. Pat. No. 4,960,716.

The further binding site may target protein antigens, carbohydrate antigens, or glycosylated proteins. For example, the variable domain can target glycosylation groups of antigens that are preferentially produced by transformed (neoplastic or cancerous) cells, infected cells, and the like (cells associated with other immune system-related disorders). In one aspect, the antigen is a tumor-associated antigen. In an exemplary aspect, the antigen is O-acetylated-GD2 or glypican-3. In another particular aspect, the antigen is one of the Thomsen-Friedenreich (TF) antigens (TFAs).

The further binding site can also exhibit specificity for a cancer-associated protein. Such proteins can include any protein associated with cancer progression. Examples of such proteins include angiogenesis factors associated with tumor growth, such as vascular endothelial growth factors (VEGFs), fibroblast growth factors (FGFs), tissue factor (TF), epidermal growth factors (EGFs), and receptors thereof; factors associated with tumor invasiveness; and other receptors associated with cancer progression (e.g., one of the HER1-HER4 receptors).

In some embodiments, the tumor antigen is HER. In a preferred embodiment, the tumor antigen is HER 2. Typically, HER2 is a specialized protein found on breast cancer cells that controls cancer growth and spread.

In a preferred embodiment, the polypeptide according to this invention may consist of: 1) a first fusion protein wherein the CH1 constant domain of an antibody is fused by its N-terminal end to the C-terminal end to a single domain antibody according to the invention (a single domain antibody directed against NKG2D) and ii) a second fusion protein wherein the CL constant domain of an antibody is fused by its N-terminal end to the C-terminal end of a single domain antibody directed against an antigen different from NKG2D. In another particular embodiment, the polypeptide may consist of i) a first fusion protein wherein the CH1 constant domain of an antibody is fused by its N-terminal end to the C-terminal end to a single domain antibody according to the invention (a single domain antibody directed against NKG2D) and ii) a second fusion protein wherein the CL constant domain of an antibody is fused by its N-terminal end to the C-terminal end of a single domain antibody directed against HER-2.

In a particular embodiment, the polypeptide of the present invention comprises i) a first single domain antibody (ET1F08 derivate) comprising a CDR1 having least 70% of identity with sequence set forth as SEQ ID NO:1, a CDR2 having at least 70% of identity with sequence set forth as SEQ ID NO:2 and a CDR3 having at least 70% of identity with sequence set forth as SEQ ID NO:3 and ii) a second single domain antibody directed against HER-2.

In a particular embodiment, the polypeptide of the present invention comprises i) a first single domain antibody (ET1F09 derivate) comprising a CDR1 having least 70% of identity with sequence set forth as SEQ ID NO:5, a CDR2 having at least 70% of identity with sequence set forth as SEQ ID NO:6 and a CDR3 having at least 70% of identity with sequence set forth as SEQ ID NO:7 and ii) a second single domain antibody directed against HER-2.

In some embodiments, the first binding site is directed against NKG2D and the further binding site is against antigens present on pathogen infected cells. Typically, antigens present on pathogen infected cells refer to all protein, peptide, polypeptide or fragments present on pathogen infected cells. The further binding site can be specific for a virus, a bacteria or parasite associated target. For example, the further binding site may be specific for a virus-associated target such as an HIV protein (Nef, Env), CMV or other viruses, such as hepatitis C virus (HCV).

Typically, the one or more further binding site may comprise one or more parts, fragments or domains of conventional chain antibodies (and in particular human antibodies) and/or of heavy chain antibodies. For example, a single domain antibody of the invention may be linked to a conventional (typically human) VH or VL optionally via a linker sequence.

In some embodiments, the polypeptides comprise a single domain antibody of the invention that is linked to an immunoglobulin domain. For example the polypeptides comprise a single domain antibody of the invention that is linked to an Fc portion (such as a human Fc). Said Fc portion may be useful for increasing the half-life and even the production of the single domain antibody of the invention. For example the Fc portion can bind to serum proteins and thus increases the half-life on the single domain antibody. In some embodiments, the at least one single domain antibody may also be linked to one or more (typically human) CH1, and/or CH2 and/or CH3 domains, optionally via a linker sequence. For instance, a single domain antibody linked to a suitable CH1 domain could for example be used together with suitable light chains to generate antibody fragments/structures analogous to conventional Fab fragments or F(ab′)2 fragments, but in which one or (in case of an F(ab′)2 fragment) one or both of the conventional VH domains have been replaced by a single domain antibody of the invention.

In some embodiments, one or more single domain antibodies of the invention may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more CH2 and/or CH3 domains of an antibody, such as from a heavy chain antibody and more typically from a conventional human chain antibody; and/or may form and Fc region, for example from IgG (e.g. from IgG1, IgG2, IgG3 or IgG4), from IgE or from another human Ig such as IgA, IgD or IgM. For example, WO 94/04678 describes heavy chain antibodies comprising a Camelid VHH domain or a humanized derivative thereof (i.e. a single domain antibody), in which the Camelidae CH2 and/or CH3 domain have been replaced by human CH2 and CH3 domains, so as to provide an immunoglobulin that consists of 2 heavy chains each comprising a single domain antibody and human CH2 and CH3 domains (but no CHI domain), which immunoglobulin has the effector function provided by the CH2 and CH3 domains and which immunoglobulin can function without the presence of any light chains.

In some embodiments, the polypeptide is as described in WO2006064136. In particular the polypeptide may consist of i) a first fusion protein wherein the CH1 constant domain of an antibody is fused by its N-terminal end to the C-terminal end to a single domain antibody according to the invention (i.e. a single antibody directed against NKG2D) and ii) a second fusion protein wherein the CL constant domain of an antibody is fused by its N-terminal end to the C-terminal end of a single domain antibody directed against an antigen different from NKG2D. In another particular embodiment, the polypeptide may consist of i) a first fusion protein wherein the CH1 constant domain of an antibody is fused by its N-terminal end to the C-terminal end to a single domain antibody according to the invention (i.e. a single antibody directed against NKG2D) and ii) a second fusion protein wherein the CL constant domain of an antibody is fused by its N-terminal end to the C-terminal end of a single domain antibody directed against an epitope, antigen, target, protein or polypeptide which is present on tumour cells.

In some embodiments, the polypeptide of the present invention comprises ET1F08 derivative as defined above. In some embodiments, the polypeptide of the present invention comprises i) a first single domain antibody (ET1F08 derivate) comprising a CDR1 having least 70% of identity with sequence set forth as SEQ ID NO:1, a CDR2 having at least 70% of identity with sequence set forth as SEQ ID NO:2 and a CDR3 having at least 70% of identity with sequence set forth as SEQ ID NO:3 and ii) a second single domain antibody which is different from the first single domain antibody. In a particular embodiment, the polypeptide of the present invention comprises i) a first single domain antibody (ET1F08 derivate) comprising a CDR1 having least 70% of identity with sequence set forth as SEQ ID NO:1, a CDR2 having at least 70% of identity with sequence set forth as SEQ ID NO:2 and a CDR3 having at least 70% of identity with sequence set forth as SEQ ID NO:3 and ii) a second single domain antibody directed against an epitope, antigen, target, protein or polypeptide of tumour cells

In some embodiments, the polypeptide of the present invention comprises ET1F09 derivative as defined above. In some embodiments, the polypeptide of the present invention comprises i) a first single domain antibody (ET1F09 derivate) comprising a CDR1 having least 70% of identity with sequence set forth as SEQ ID NO:5, a CDR2 having at least 70% of identity with sequence set forth as SEQ ID NO:6 and a CDR3 having at least 70% of identity with sequence set forth as SEQ ID NO:7 and ii) a second single domain antibody which is different from the first single domain antibody. In a particular embodiment, the polypeptide of the present invention comprises i) a first single domain antibody (ET1F09 derivate) comprising a CDR1 having least 70% of identity with sequence set forth as SEQ ID NO:5, a CDR2 having at least 70% of identity with sequence set forth as SEQ ID NO:6 and a CDR3 having at least 70% of identity with sequence set forth as SEQ ID NO:7 and ii) a second single domain antibody directed against an epitope, antigen, target, protein or polypeptide of tumour cells.

According to the invention, the single domain antibodies and polypeptides of the invention may be produced by conventional automated peptide synthesis methods or by recombinant expression. General principles for designing and making proteins are well known to those of skill in the art.

The single domain antibodies and polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The single domain antibodies and polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the single domain antibodies and polypeptides of the invention, it would be necessary to employ vectors comprising polynucleotide molecules for encoding the single domain antibodies and polypeptides of the invention. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (e.g., a single domain antibody). Thus, a promoter nucleotide sequence is operably linked to a given DNA sequence if the promoter nucleotide sequence directs the transcription of the sequence.

### Method for Detecting NK Cells

The single domain antibodies or polypeptides according to the invention are suitable for detecting NK cells in a biological sample obtained from a subject.

The term “biological sample” is used herein in its broadest sense. A biological sample is generally obtained from a subject. A sample may be of any biological tissue or fluid with which the detection of NK cells according to the invention may be assayed. Frequently, a sample will be a “clinical sample”, i.e., a sample derived from a patient. Such samples include, but are not limited to, bodily fluids which may or may not contain cells, e.g., blood, synovial fluid, saliva, tissue or fine needle biopsy samples, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample, or proteins extracted from the sample. Processing of a biological sample may involve one or more of: filtration, distillation, extraction, concentration, inactivation of interfering components, addition of reagents, and the like.

In a particular embodiment, the biological sample is peripheral blood mononuclear cell (PBMC).

Typically, the presence of NK cells in the PBMC of the patient consists in detecting the presence and/or absence of some specific cell surface markers. In the context of the invention, the specific cell surface marker of NK cells is NKG2D. Standard methods for detecting the expression of specific surface markers at NK cell surface are well known in the art. Typically, the step consisting of detecting the presence of NK cells involves in the use of single domain antibodies or polypeptides according to the invention. Typically, providing a biological sample obtained from a subject, contacting the biological sample with the single domain antibodies or polypeptides according to the invention. The single domain antibodies or polypeptides according to the invention are typically labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the single domain antibodies or polypeptides according to the invention, is intended to encompass direct labelling of the single domain antibodies or polypeptides according to the invention by coupling (i.e., physically linking) a detectable substance, such as a fluorophore [e.g. fluorescein isothiocyanate (FITC) or phycoerythrin (PE) or Indocyanine (Cy5)]) or a radioactive agent to single domain antibodies or polypeptides according to the invention, as well as indirect labelling of the probe or antibody by reactivity with a detectable substance. The single domain antibodies or polypeptides according to the invention may be labelled with a radioactive molecule by any method known in the art. For example radioactive molecules include but are not limited radioactive atom for scintigraphic studies such as I¹²³, I¹²⁴, In¹¹¹, Re¹⁸⁶, Re¹⁸⁸. In some embodiments, the single domain antibodies or polypeptides according to the invention are already conjugated to a fluorophore (e.g. FITC-conjugated and/or PE-conjugated).

The aforementioned assays typically involve in the binding of the single domain antibodies or polypeptide of the invention to a solid support. The solid surface could a microtitration plate coated with the single domain antibodies or polypeptide of the invention. After incubation of the NK cell sample, NK cells specifically bound to the the single domain antibodies or polypeptide of the invention may be detected. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.).

In a particular embodiment, the biological sample is a tissue sample.

As used herein, the term “tissue sample” refers to a sample that is typically made up of a collection of biological cells and includes, but is not limited to, for example, biopsy samples, autopsy samples, surgical samples, cell smears, cell concentrates and cultured cells fixed on a support. Typically, the tissue sample generally includes any material for which microscopic examination of samples of the material prepared on microscope slides is desirable. The tissue sample may be collected for diagnostic, research, teaching or other purposes. The sample may be of any biological tissue. Examples of tissue samples include, but are not limited to, tissue sections of brain, adrenal glands, colon, small intestines, stomach, heart, liver, skin, kidney, lung, pancreas, testis, ovary, prostate, uterus, thyroid and spleen. The “tissue sample” as used herein may be sections of tissues that are either fresh, or frozen, or fixed and embedded. For example, tissue samples for histological examination are embedded in a support medium and moulded into standardized blocks. Paraffin wax is a known and commonly-used as a support medium, however it will be appreciated that other support media, including but not limited to, TissueTek O.C.T., manufactured by Sakura Finetek, ester, microcrystalline cellulose, bees wax, resins or polymers, such as methacrylates, may also be used as support media. Suitable resins and polymers, including Araldite 502 Kit, Eponate12™, Kit, and Glycol Methacrylate (GMA) Kit, are available from Ted Pella, Inc., Redding, Calif.

In some embodiments, the tissue sample is a tumor sample. A “tumor sample” is a sample containing tumor material e.g. tissue material from a neoplastic lesion taken by aspiration or puncture, excision or by any other surgical method leading to biopsy or resected cellular material, including preserved material such as fresh frozen material, formalin fixed material, paraffin embedded material and the like. Such a biological sample may comprise cells obtained from a patient.

The tissue sample can be subjected to a variety of well-known post-collection preparative and storage techniques (e.g., fixation, storage, freezing, etc.) prior to determining the cell densities. Typically the tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (IHC) (using an IHC automate such as BenchMark® XT or Autostainer Dako, for obtaining stained slides).

Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the single domain antibodies or polypeptides of the present invention, v) rinsing said sections, vi) incubating said section with a secondary antibody typically biotinylated and vii) revealing the antigen-antibody complex typically with avidin-biotin-peroxidase complex. Accordingly, the tissues sample is firstly incubated with the single domain antibodies or polypeptides according to the invention. After washing, the labeled antibodies that are bound to NKG2D are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems. For example, one or more labels can be attached to the single domain antibodies and polypeptides of the present invention, thereby permitting detection of the NKG2D. Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the single domain antibodies or polypeptides of the invention can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample, or the absolute number of cells positive for the maker of interest, or the surface of cells positive for the maker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms and tissue recognition pattern (e.g. Aperio Spectrum Software, Automated QUantitatative Analysis platform (AQUA® platform), or Tribvn with Ilastic and Calopix software), and other standard methods that measure or quantitate or semi-quantitate the degree of staining; see e.g., U.S. Pat. No. 8,023,714; U.S. Pat. No. 7,257,268; U.S. Pat. No. 7,219,016; U.S. Pat. No. 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. For example, the amount can be quantified as an absolute number of cells positive for the maker of interest. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above described), ii) proceeding to digitalisation of the slides of step i). by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity or the absolute number of stained cells in each unit whereby the number or the density of cells stained of each unit is assessed.

The term “subject” refers to a mammal, typically a human. In a particular embodiment, a subject refers to any subject (typically human) having or is susceptible to be afflicted with cancer, infectious diseases or autoimmune diseases.

### Method of Treatment

In some embodiments, the present invention relates to a method for treating tumours in a subject in need thereof comprising a step of administering to said subject a therapeutically effective amount of a single domain antibody or a polypeptide according the invention.

Tumors to be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued. Examples of cancers that may be treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangio sarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a further embodiment, the present invention relates to a method for treating infectious diseases in a subject in need thereof comprising a step of administrating to said subject a therapeutically effective amount of a single domain antibody or polypeptides according to the invention.

As used herein, the term “infectious disease” is intended to encompass any disease which results from an infection mediated by a virus, a bacteria or a parasite. Therefore the term includes but is not limited to infection with virus such as human immunodeficiency virus, Hepatitis B virus, hepatitis C virus, with parasites such as Plasmodium Falciparum (causative agent for Malaria), or with bacteria such as mycobacterium tuberculosis.

In a further embodiment the present invention relates to a method for treating autoimmune diseases.

As used herein, the term “autoimmune diseases” has its general meaning in the art and refers to when the immune system attacks self-tissue. Autoimmune diseases include but are not limited to Addison's disease, ankylosing spondylitis, aplastic anemia, autoimmune hemolytic anemia, autoimmune hepatitis, coeliac disease, Crohn's disease, dermatomyositis, Goodpasture's syndrome, Graves' disease, Guillain-Barre syndrome, Hashimoto's disease, idiopathic leucopenia, idiopathic thrombocytopenic purpura, insulin dependent diabetes mellitus (Type 1 diabetes), male infertility, mixed connective tissue disease, multiple sclerosis (MS), myasthenia gravis, pemphigoid, pemphigus vulgaris, pernicious anemia, phacogenic uveitis, primary biliary cirrhosis, primary myxoedema, Reiter's syndrome, rheumatoid arthritis (RA), scleroderma, Sjogren's syndrome, stiff man syndrome, systemic lupus erythematosus (SLE), thyrotoxicosis, ulcerative colitis, and Wegener's granulomatosis.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

By a “therapeutically effective amount” is meant a sufficient amount of the single domain antibody of the invention or the polypeptide of the invention to treat the disease (e.g. cancer) at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The single domain antibodies and polypeptides of the invention or the polypeptide of the present invention may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: A: Specificity of sdAb ET1F8 and ET2F9 were tested by ELISA on plate coated with BSA, MICA-Fc, human NKG2D or murin NKG2D as described in mat and meth. Experiment was done in triplicate and results analyzed by One way ANOVA (p<0.0001). B: affinité of sdAb ET1F8 and ET2F9 were tested by flow cytometry on HEK-293 transfected cells (Kd sdAb F8 de 110 nM et sdAb F9 625 nM) and by ELISA on plate coated with Fc-NKG2D. Dose-response curves were treated by nonlinear regression analysis (one site total binding equation) using Prism software (GraphPad Software) (EC50 sdAb F8 de 6.2 nM et sdAB F9 de 44 nM).

FIG. 2: A: Competitive phage-sdAbs assay: competition between of sdAb F8 and F9 were tested by flow cytometry on HEK-293 transfected cells. Cells were first incubated with 20 μM sdAbs then with phage containing supernatants. Cells were stained with PE-conjugated anti M13 mab. Experiment was done in duplicate. Negative control correspond to cells stained without sdAbs or phages. B: competition between of sdAb F8 or F9 and MICA-Fc and mab NKG2D were tested by flow cytometry on HEK-293 transfected cells as described in mat & meth. Binding was followed staining cells with PE-conjugated goat anti-human antibody for MICA-Fc detection and PE-conjugated goat anti-mouse antibody for monoclonal Human NKG2D antibody detection. Dose-response curves were treated by nonlinear regression (log inhibitor vs response equation) using Prism software (GraphPad Software).

FIG. 3: A: BsfAb construction. B: BsfAb ET1F8 and ET2F9 were purified from E. coli DH5a transformed culture as described in mat and meth, by affinity chromatography on IgG-CH1 matrix and) LC-CKappa matrix (Capture select) after periplasmic extraction by osmotic shock. bsfAbs production was controled by 4-15% stain free polyacrylamide gel (BIO-RAD) under reducing (R) and nonreducing (NR) conditions. 5 μg of each bsfAb and 10 μl of stain free ladder were loaded. C: bsfAb binding activity was assessed by flow cytometry on HEK-293 transfected cells and BT-474 cells. Bound antibody was detected with anti-6His tag antibody ( 1/1000 Novagen) followed by PE-conjugated goat anti-mouse ( 1/400 Santa Cruz Biotech). Dose-response curves were treated by nonlinear regression analysis (one site total binding equation) using Prism software (GraphPad Software).

FIG. 4: bsFab-dependent cytolitic activity of NK cells toward BT474 breast cancer cells. Experiments were done in triplicate with 3 different donors. Target cell viability was quantified using CellTiter-Glo viability assay (Promega). Dose-response curves were treated by nonlinear regression analysis using Prism software (GraphPad Software). Data were expressed as mean±SEM.

FIG. 5: Measure of IFN-g production by NK92 CD16 cells prestimulated by IL2 ON. Total concentration of IFN-γ was quantified in the supernatants of NK cells using enzyme-linked immunosorbent assay (ELISA) kits from eBioscience, following incubation with anti-NKG2D, anti-2B4 or purified anti-NKG2D sdAb. Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 and cells stimulated with phorbol-12-myristate-13-acetate (PMA) (2.5 μg/ml) and ionomycin (0.5 μg/ml) (Sigma-Aldrich) were used as a positive control.

FIG. 6: Measure of IFN-g production by fresh NK cells prestimulates ON by 1000 UI/ml IL2. Total concentration of IFN-γ was quantified in the supernatants of NK cells using enzyme-linked immunosorbent assay (ELISA) kits from eBioscience, following incubation with anti-NKG2D, anti-2B4 or unpurified anti-NKG2D sdAb. Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 was used as positive control.

FIG. 7: Measure of IFN-g production by fresh NK cells prestimulated ON by 1000 UI/ml IL2. Total concentration of IFN-γ was quantified in the supernatants of NK cells using enzyme-linked immunosorbent assay (ELISA) kits from eBioscience, following incubation with anti-NKG2D, anti-2B4 or unpurified anti-NKG2D sdAb. Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 was used as positive control.

FIG. 8: Detection of CD107a by flow cytometry on fresh isolated NK cells prestimulated by 1000 UI/ml IL-2. Degranulating NK cell were determined by flow cytometry as the percentage of CD107a positive NK cells in response to NKG2D or NKG2D/2B4 engagement with anti-NKG2D, anti-2B4 antibodies or unpurified anti-NKG2D sdAb. Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 was used as positive control.

FIG. 9: Detection of CD107a by flow cytometry on fresh PBMC (NK cells gates) prestimulated ON by 1000 UI/ml IL-2. Degranulating NK cell were determined as the percentage of CD107a positive cells in the gate CD3⁻CD56⁺ NK cells in response to NKG2D or NKG2D/2B4 engagement with anti-NKG2D, anti-2B4 antibodies or unpurified anti-NKG2D sdAb. Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 was used as positive control.

### EXAMPLES

### Example 1

Material & Methods:

Cells Lines

BT-474 and HEK-293 cell lines were purchased from ATCC and grown as recommended by the manufacturer.

Human peripherical blood mononuclear cells (PBMC) were isolated from blood of healthy donors (collected in 40 ml CLP bags containing citrate phosphate dextrose as anticoagulant and no additive from Etablissement Français du Sang, Marseille, France) by Ficoll LSM1077 (PAA) gradient centrifugation method. NK cells were isolated by depleting non-NK cells using the NK cell isolation kit (Miltenyi Biotec) as described by the manufacturer.

For transfection assay, HEK/293T were co-transfected with NKG2D and DAP10 DNA (OriGene) using Lipofectamine 2000 (Invitrogen), following the recommendation of the manufacturer.

Cell surface expression of NKG2D on transfected HEK cells was analyzed by flow cytometry after labeling with an PE or APC-conjugated anti-CD314 (Miltenyi).

Llama Immunization and Library Construction:

llama (Lama glama) was immunized with recombinant human NKG2D/Fc chimera (R&D) and VHH library constructions were obtained as previously published (26, 27).

Selection of Phage-sdAbs

Phage-sdAbs were selected using protocoles previously described (3) using a two round strategy.

In a first round phages were selected using magnetic epoxy beads (Dynabeads, invitrogen) coated with antigen NKG2D/Fc (R&D system/clone 149810) during 48 h at 4° C. using conditions recommended in the manufacturer's protocol. A second round of masked selection was performed on NKG2D-DAP10 co-transfected HEK293T cells (40 to 50 millions) to avoid selection against Fc domain, including a depletion step (phages depleted on HEK).

Monoclonal Phage-sdAb and sdAb Production in Microtiter Plate and Flow Cytometry-Based Screening Assay

Phage-sdAbs or sdAb were produced using protocols previously described (27) and Phage or sdAb containing supernatants were tested for binding by flow cytometry as previously described (29) on NKG2D-DAP10 co-transfected HEK293T cells. Phage-sdAb were labeled with PE-conjugated M13 Major Coat Protein antibody (TEBU) and sdAb were incubated with a 1/500 dilution of anti-6His tag antibody (Novagen) followed by an incubation with a 1/400 dilution of a PE-conjugated goat anti-mouse antibody (Santa Cruz).

Profiles were compared to binding on non-transfected HEK293T cells to consider ratio of mean.

Production and Purification of sdAbs

SdAbs were produced and purified by metal affinity chromatography as described (27). Purity was controled with unstained gel 4-15% acrylamid (BIORAD) and western blot using a 1/5000 dilution of HRP-conjugated anti-6His tag antibody (Miltenyi).

Enzyme Linked Immunosorbent Assay (ELISA)

Human or murin Fc-NKG2D or MICA-Fc chimera proteins (R&D Systems), 50 μL at 7 μg/mL, were incubated in each well on Maxisorp plate (Nunc) overnight at 4° C.

Wells were then saturated with PBS/5% BSA before the incubation with 50 μl of sdAb (3 μMin PBS/2% BSA). Antibodies were then incubated with mouse anti-his HRP mAb (1:5000) (Miltenyi Biotec) and wells control with HRP-conjugated goat anti-mouse antibody (Jackson Immunoresearch lab) ( 1/10000) or HRP-conjugated goat anti-human antibody) ( 1/1000) and finally revealed using Substrat TMB SureBlue (KPL).

Each step was performed in 50 μl for 1 h at room temperature with shaking and followed by 3 washes with 0.1% Tween PBS and 3 washes with PBS. Absorbance was measured at 650 nm on TECAN Infinite M1000.

Flow Cytometry Assay

Experiments were performed at 4° C. with rocking in 1% BSA PBS. Typically, 3×105 cells in 50 μl buffer were distributed in 96-well microtiter plates as described (3). Fluorescence measurement was performed with a MACS-Quant cytometer (Miltenyi) and results were analyzed with the MACS-Quant software.

For competitive phage-sdAbs assay, plates were incubated with 25 μl/well of sdAbs 20 μM for 1 h.

Then 25 μl of phage-containing supernatants (10×) in 2% BSA PBS were added to each well. Plates were incubated again for 1 h. After three washes in 1% BSA PBS, plates were incubated for 1 h with PE-conjugated anti-M13 mAb at 1/100 (Santa Cruz) for phage-sdab detection. Fluorescence measurement was performed after three washes in 1% BSA PBS. Negative (secondary antibody only) controls were carried out.

For competitive sdAbs assay with MICA-Fc or monoclonal Human NKG2D antibody (R&D System clone 149810), plates were incubated with 40 μl/well of sdAbs from 0.03 pM to 100 μM for 1 h.

Then 10 μl of MICA-Fc or monoclonal Human NKG2D antibody (50 μg/ml) were added. Plates were incubated again for 1 h. After three washes in 1% BSA PBS, plates were incubated for 1 h with PE-conjugated goat anti-human antibody ( 1/100) (Beckman) or PE-conjugated goat anti-mouse antibody ( 1/400) (Santa Cruz Biotech) to detect respectively MICA-Fc or monoclonal Human NKG2D antibody.

Production and Purification of bsfAb

E. coli DH5α were transformed with HER2-c7b×NKG2D-ET1F8/14aHER2×CD16 or HER2-c7b×NKG2D-ET2F9/14aHER2×CD16 plasmid and grown overnight in LB medium supplemented with 100 μg/ml ampicillin and 2% glucose, then diluted 1:100 and grown in 3.2 1 LB medium supplemented with 100 μg/ml ampicillin cultures at 30° C. After reaching an optical density of 2, the temperature was decreased down to 10° C. for 1 h and the production was induced with 100 μM IPTG for 70 h at 10° C. BsFab purification was done by affinity chromatography as described previously (30). BsFab purity and integrity were controlled by unstained gel 12% acrylamid (BIORAD) and western-blotting using mouse anti-his HRP mAb (1:5000) (Miltenyi Biotec) and mouse anti-flag M2 mAb (1:5000) (Sigma Aldrich). Protein concentrations were determined using a Direct Detect spectrometer (Merck Millipore).

Binding Activity of bsfAb Assessed by Flow Cytometry with Transfected HEK-293 and BT-474

BsfAb binding activity was analysed using HEK-293 and BT-474. Cells were incubated with 50 μl of diluted antibodies from 17 nM to 3 μM for 1 h at 4° C. with shaking. Bound antibody was detected using mouse anti-his monoclonal antibody ( 1/1000; Novagen) followed by incubation with PE-conjugated goat anti-mouse IgG F(ab′)2 ( 1/400; santa cruz biotech).

ADCC Assay

Target cells (BT474 5×103 cells/well) were mixed with 5×104 freshly isolated human NK cells (effector/target ratio: 10:1). Variable concentrations of bsFabs were added to the cells in a final volume of 200 μL. All procedures were done in triplicate with different donors. Following overnight incubation at 37° C., target cell viability was quantified with CellTiter-Glo viability assay (Promega) according to manufacturer's protocol. Luminescence was measured on TECAN Infinite M1000. Dose-response curves were treated by nonlinear regression analysis using Prism software (GraphPad Software). Data were expressed as mean±SEM.

Results:

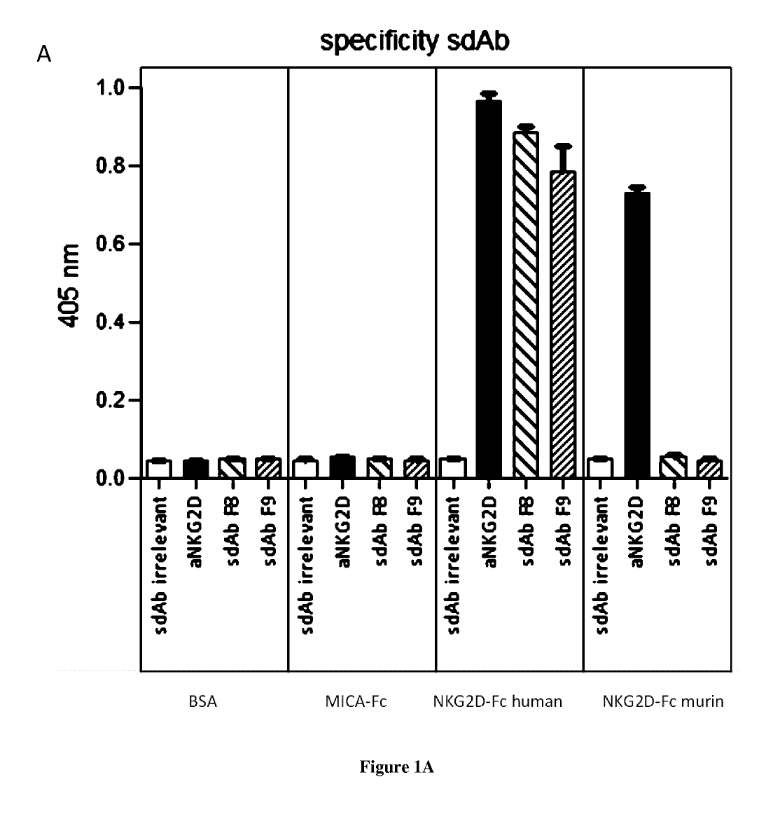

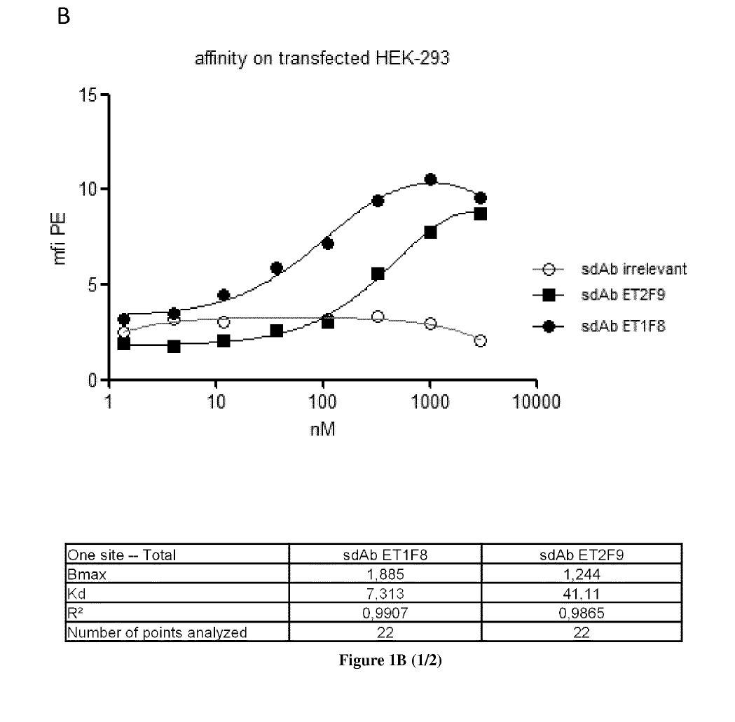

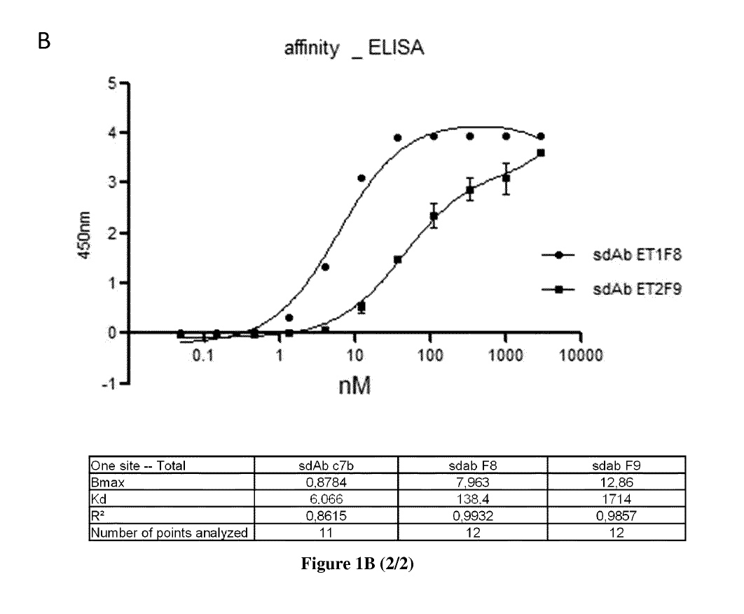

Inventors have demonstrated the specificity of sdAb ET1F8 and ET2F9 for NKGD2-Fc human by ELISA (FIG. 1A), on plate coated with BSA, MICA-Fc, human NKG2D or murin NKG2D as described in material and method.

The affinity of sdAb ET1F8 and ET2F9 were tested by flow cytometry on HEK-293 transfected cells: Kd of sdAb F8 is 110 nM and Kd of sdAb F9 is 625 nM (FIG. 1B). Inventors obtained dose-response curves which were treated by nonlinear regression analysis (one site total binding equation) using Prism software (GraphPad Software) (EC50 of sdAb F8 is 6.2 nM and EC50 of sdAB F9 is 44 nM).

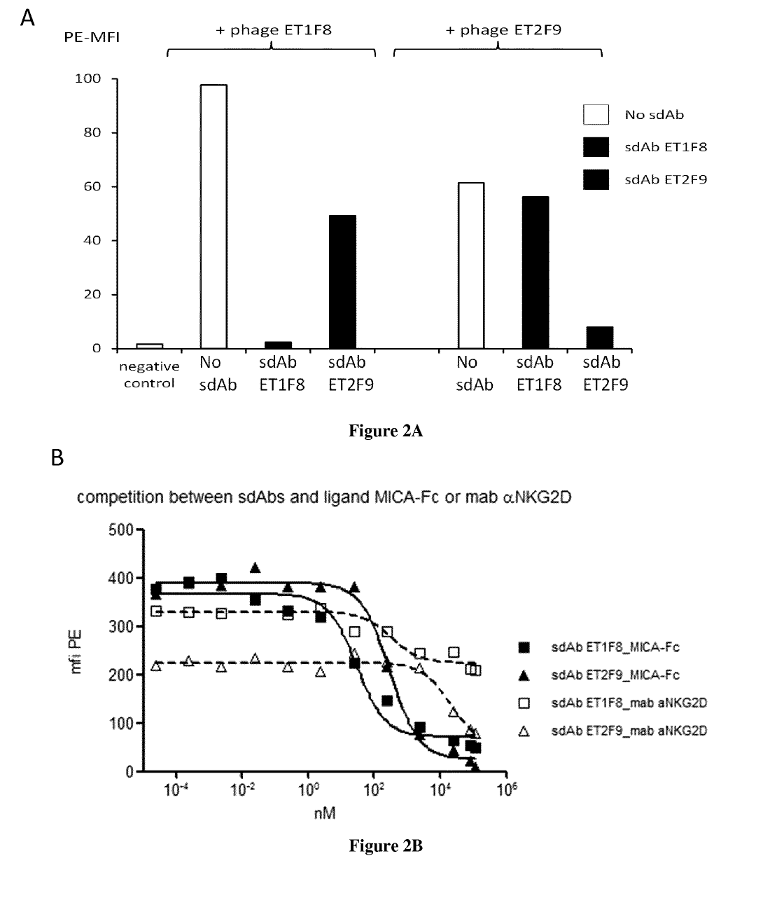

The competition between sdAb F8 and F9 were tested by flow cytometry on HEK-293 transfected cells. Cells were first incubated with 20 μM sdAbs then with phage containing supernatants. Cells were stained with PE-conjugated anti M13 mab. Experiment was done in duplicate. Negative control correspond to cells stained without sdAbs or phages (FIG. 2A). Competition between of sdAb F8 or F9 and MICA-Fc and mab NKG2D were tested by flow cytometry on HEK-293 transfected cells as described in material & method (FIG. 2B). Binding was followed staining cells with PE-conjugated goat anti-human antibody for MICA-Fc detection and PE-conjugated goat anti-mouse antibody for monoclonal Human NKG2D antibody detection. Dose-response curves were treated by nonlinear regression (log inhibitor vs response equation) using Prism software (GraphPad Software).

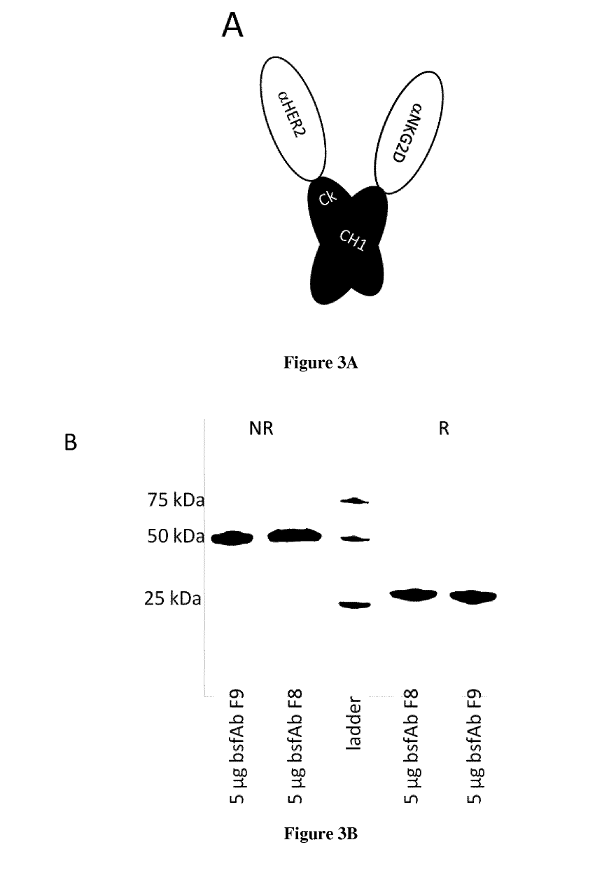

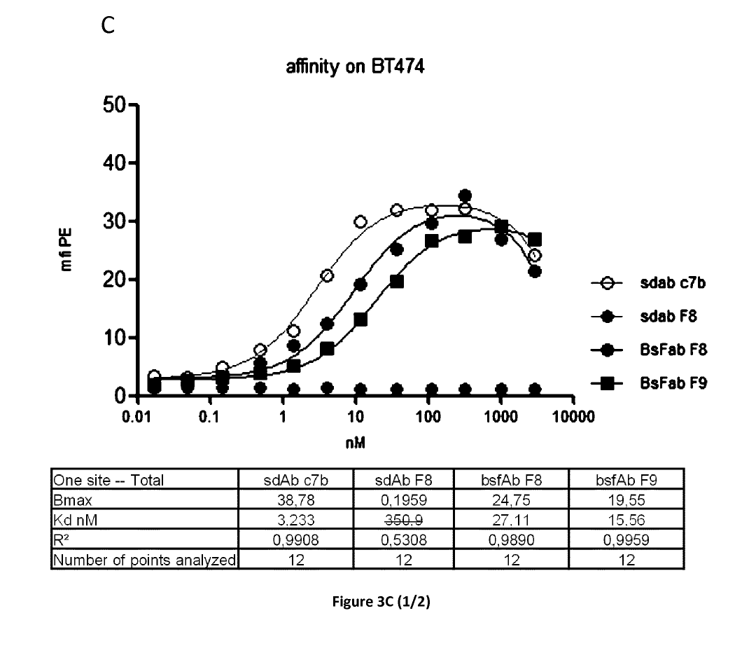

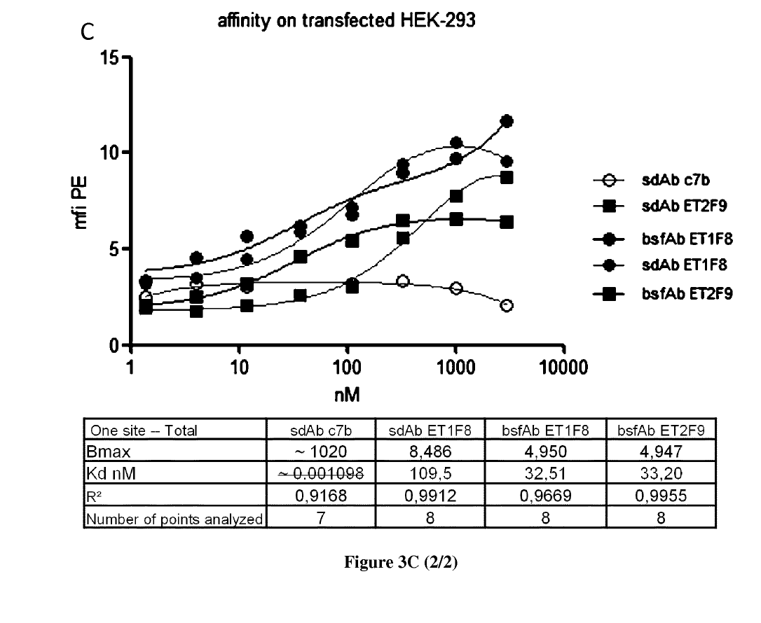

BsfAb ET1F8 and ET2F9 were purified from E. coli DH5a transformed culture as described in material and method, by affinity chromatography on IgG-CH1 matrix and LC-CKappa matrix (Capture select) after periplasmic extraction by osmotic shock. bsfAbs production was controlled by 4-15% stain free polyacrylamide gel (BIO-RAD) under reducing (R) and nonreducing (NR) conditions. 5 μg of each bsfAb and 10 μl of stain free ladder were loaded (FIG. 3B). The bsfAb binding activity was assessed by flow cytometry on HEK-293 transfected cells and BT-474 cells (FIG. 3C). The bound antibody was detected with anti-6His tag antibody ( 1/1000 Novagen) followed by PE-conjugated goat anti-mouse ( 1/400 Santa Cruz Biotech). Dose-response curves were treated by nonlinear regression analysis (one site total binding equation) using Prism software (GraphPad Software).

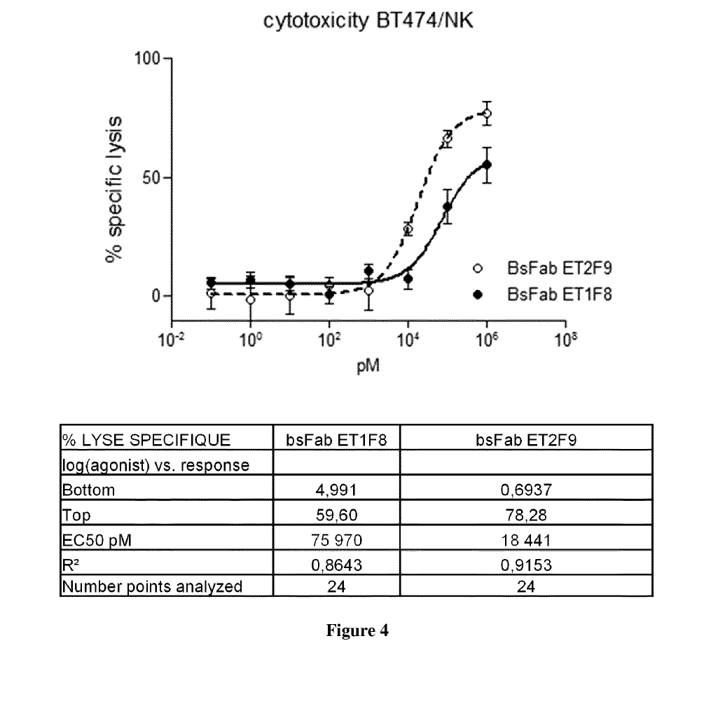

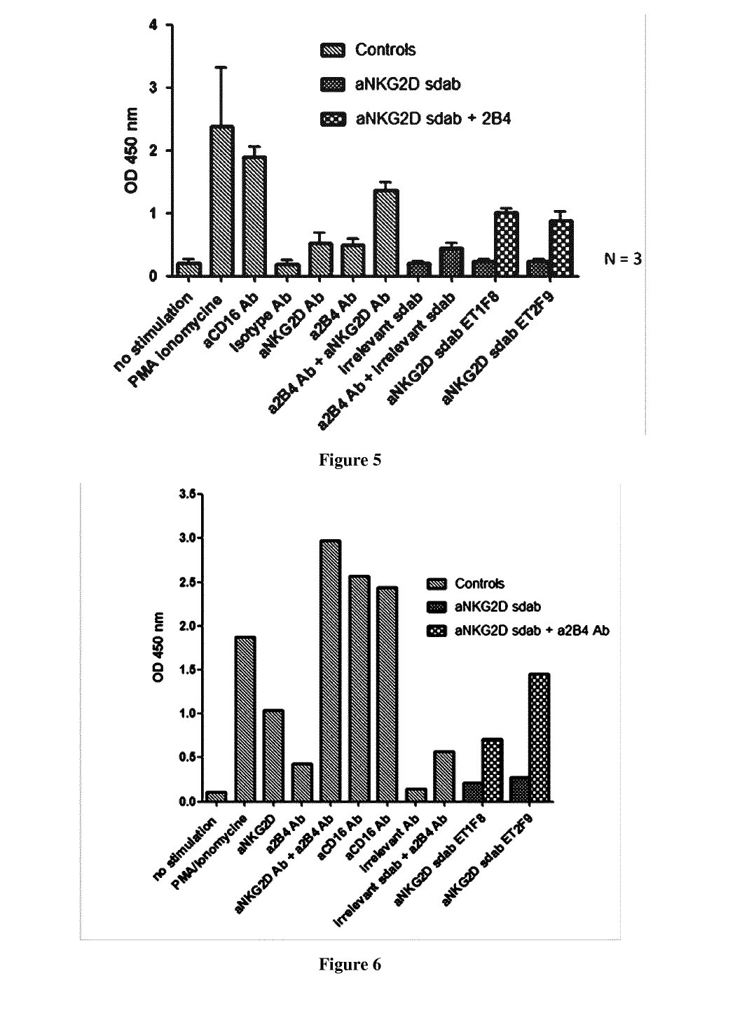

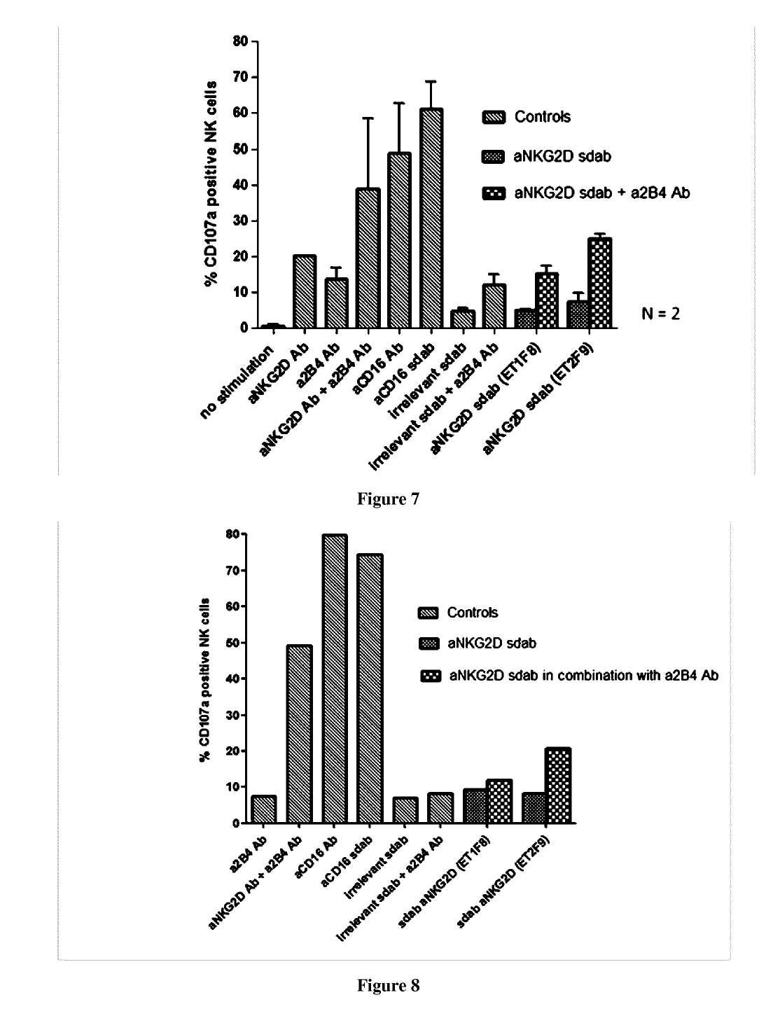

The bsFab-dependent cytolitic activity of NK cells toward BT474 breast cancer cells were tested and FIG. 4 shows that these antibodies increase BT474 breast cancer cells lysis.

These results show that the antibodies according to the invention are able to enhance NK cells activity toward the tumor cells. Thus, these results allow new promising therapeutics approaches to treat cancer, infectious diseases or autoimmune diseases.

### Example 2: Analysis of IFN-γ Production in IL2 Pre-Activated CD16-Transfected NK 92 Cells in Response to NKG2D Engagement

Total concentration of IFN-γ was quantified in the supernatants of NK cells using enzyme-linked immunosorbent assay (ELISA) kits from eBioscience, following incubation with anti-NKG2D, anti-2B4 or purified anti-NKG2D sdAb.

Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 and cells stimulated with phorbol-12-myristate-13-acetate (PMA) (2.5 μg/ml) and ionomycin (0.5 μg/ml) (Sigma-Aldrich) were used as a positive control.

Purified anti-NKG2D sdAbs (ET1F8 and ET2F9), anti-CD16.21 sdAb (positive control) and an irrelevant sdab (2.5 μg/ml) were captured on plate previously coated with anti-6HIS (5 μg/mL) in combination with anti-HIS isotype control or monoclonal anti-2B4 (5 μg/ml). CD16 transfected NK 92 cells were prestimulated O/N with 1000 UI/mL IL2 and then added to the activation plate at 0.15×106 cells/well for 2 hours, 37° C., CO2 5%.

### Example 3: Analysis of IFN-g Production in IL2 Pre-Activated Human NK Cells in Response to NKG2D Engagement

Total concentration of IFN-γ was quantified in the supernatants of NK cells using enzyme-linked immunosorbent assay (ELISA) kits from eBioscience, following incubation with anti-NKG2D, anti-2B4 or unpurified anti-NKG2D sdAb.

Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 was used as positive control.

Anti-NKG2D sdAbs (ET1F8 and ET2F9), anti-CD16.21 sdAb (positive control) and an irrelevant sdab from soluble lysates of E. coli were captured on plate bound anti-6HIS (5 μg/mL) in combination with anti-HIS isotype control or monoclonal anti-2B4 (5 μg/ml). NK cells, isolated by negative selection from human PBMC (Human NK cell isolation kit—Miltenyi) were prestimulated O/N with 1000 UI/mL IL2 and then added to the activation plate at 0.15×10⁶ cells/well for 2 hours, 37° C., CO2 5%.

### Example 4: Analysis of Degranulating NK Cells in Response to NKG2D Engagement in IL2 Pre-Activated Human NK Cells

Degranulating NK cell were determined by flow cytometry as the percentage of CD107a positive NK cells in response to NKG2D or NKG2D/2B4 engagement with anti-NKG2D, anti-2B4 antibodies or unpurified anti-NKG2D sdAb.

Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 was used as positive control.

Anti-NKG2D sdAbs (ET1F8 and ET2F9), anti-CD16.21 sdAb and an irrelevant sdab from soluble lysates of E. coli were captured on plate bound anti-6HIS (5 μg/mL) in combination with anti-HIS isotype control or monoclonal anti-2B4 (5 μg/ml). NK cells, isolated by negative selection from human PBMC, were prestimulated O/N with 1000 UI/mL IL2 and then added to the activation plate at 0.15×106 cells/well for 2 hours, 37° C., CO2 5%. Anti CD107a-FITC, anti-CD56YY were used for staining NK cells.

### Example 5: Analysis of Degranulating NK Cells in Response to NKG2D Engagement in IL2 Pre-Activated Human PBMC

Degranulating NK cell were determined as the percentage of CD107a positive cells in the gate CD3-CD56+NK cells in response to NKG2D or NKG2D/2B4 engagement with anti-NKG2D, anti-2B4 antibodies or unpurified anti-NKG2D sdAb.

Monoclonal anti-NKG2D (149810-R&D) (5 μg/ml) was coated O/N at 4° C. on a plate in combination with an isotype control or monoclonal anti-2B4 (C1.7-Biolegend) (5 μg/ml). CD16 engagement with plate-bound monoclonal 3G8 was used as positive control.

Anti-NKG2D sdAbs (ET1F8 and ET2F9), anti-CD16.21 sdAb and an irrelevant sdab from soluble lysates of E. coli were captured on plate bound anti-6HIS (5 μg/mL) in combination with anti-HIS isotype control or monoclonal anti-2B4 (5 μg/ml). Human PBMC were prestimulated O/N with 1000 UI/mL IL2 and then added to the activation plate at 106 cells/well for 2 hours, 37° C., CO2 5%.

### REFERENCES

Throughout this application, various references describe the state of the art to which this invention pertains. The disclosures of these references are hereby incorporated by reference into the present disclosure.
1. Cerwenka et al 2001, Natural killer cells viruses and cancer. Nature Reviews Immunology 1, 41-49 2001
2. Moretta, A. et al. Activating receptors and coreceptors involved in human natural killer cell-mediated cytolysis. Annu. Rev. Immunol. 19, 197-223 (2001).
3. A comprehensive review of the NKp30, NKp44 and NKp46 receptors and their potential role in tumour recognition.
4. Long, E. O. Regulation of immune responses through inhibitory receptors. Annu. Rev. Immunol. 17, 875-904 (1999).
5. Ravetch, J. V. & Lanier, L. L. Immune inhibitory receptors. Science 290, 84-89 (2000).
6. Leiden, J. M., Karpinski, B. A., Gottschalk, L. & Kornbluth, J. Susceptibility to natural killer cell-mediated cytolysis is independent of the level of target cell class I HLA expression. J. Immunol. 142, 2140-2147 (1989).
7. Nishimura, M. I., Stroynowski, I., Hood, L. & Ostrand-Rosenberg, S. H-2Kb antigen expression has no effect on natural killer susceptibility and tumorigenicity of a murine hepatoma. J. Immunol. 141, 4403-4409 (1988).
8. Pena, J. et al. Natural killer susceptibility is independent of HLA class I antigen expression on cell lines obtained from human solid tumors. Eur. J. Immunol. 20, 2445-2449 (1990).
9. Litwin, V., Gumperz, J., Parham, P., Phillips, J. H. &Lanier, L. L. Specificity of HLA class I antigen recognition by human NK clones: evidence for clonal heterogeneity, protection by self and non-self alleles, and influence of the target cell type. J. Exp. Med. 178, 1321-1336 (1993).
10. Lanier, L. L. On guard activating NK cell receptors. Nature Immunol. 2, 23-27 (2001).
11. Bauer, S. et al. Activation of natural killer cells and T cells by NKG2D, a receptor for stress-inducible MICA. Science 285, 727-730 (1999).
12. Houchins, J. P., Yabe, T., McSherry, C. & Bach, F. H. DNA sequence analysis of NKG2, a family of related cDNA clones encoding type II integral membrane proteins on human natural killer cells. J. Exp. Med. 173, 1017-1020 (1991).
13. Wu, J., Cherwinski, H., Spies, T., Phillips, J. H. & Lanier, L. L. DAP10 and DAP12 form distinct, but functionally cooperative, receptor complexes in natural killer cells. J. Exp. Med. 192, 1059-1068 (2000).
14. Wu, J. et al. An activating receptor complex on natural killer and T cells formed by NKG2D and DAP10. Science 285, 730-732 (1999).
15. Wilson, M. J., Lindquist, J. A. & Trowsdale, J. DAP12 and KAP10 (DAP10)—novel transmembrane adapter proteins of the CD3ζ family. Immunol. Res. 22, 21-42 (2000).
16. Li, P. et al. Complex structure of the activating immunoreceptor NKG2D and its MHC class I-like ligand MICA. Nature Immunol. 2, 443-451 (2001).
17. Marine Champsaur and Lewis L. Lanier Effect of NKG2D ligand expression on host immune responses Immunol Rev. 2010 May; 235(1): 267-285
18. Muyldermans S (2013) Nanobodies: natural single-domain antibodies. Annual review of biochemistry 82:775-797.
19. Perez J M, et al. (2001) Thermal unfolding of a llama antibody fragment: a two-state reversible process. Biochemistry 40(1):74-83.
20. Gueorguieva D, et al. (2006) Identification of single-domain, Bax-specific intrabodies that confer resistance to mammalian cells against oxidative-stress-induced apoptosis. FASEB journal: official publication of the Federation of American Societies for Experimental Biology 20(14):2636-2638.
21. Muyldermans S (2001) Single domain camel antibodies: current status. Journal of biotechnology 74(4):277-302.
22. De Genst E, et al. (2006) Molecular basis for the preferential cleft recognition by dromedary heavy-chain antibodies. Proceedings of the National Academy of Sciences of the United States of America 103(12):4586-4591.
23. Irannejad R, et al. (2013) Conformational biosensors reveal GPCR signalling from endosomes. Nature 495(7442):534-538.
24. Staus D P, et al. (2014) Regulation of beta2-Adrenergic Receptor Function by Conformationally Selective Single-Domain Intrabodies. Molecular pharmacology 85(3):472-481.
25. Behar G., Chames P., Teulon I., Cornillon A., Alshoukr F., Roquet F., Pugniere M., Teillaud J. L., Gruaz-Guyon A., Pelegrin A., Baty D. Llama single-domain antibodies directed against nonconventional epitopes of tumor-associated carcinoembryonic antigen absent from nonspecific cross-reacting antigen. 2009 FEBS J. 276, 3881-3893
26. Alvarez-Rueda N., Behar G., Ferre V., Pugniere M., Roquet F., Gastinel L., Jacquot C., Aubry J., Baty D., Barbet J., Birkle S. Generation of llama single-domain antibodies against methotrexate, a prototypical hapten. (2007) Mol. Immunol. 44, 1680-1690
27. Masked Selection: A Straightforward and Flexible Approach for the Selection of Binders Against Specific Epitopes and Differentially Expressed Proteins by Phage Display*Klervi Even-Desmureaux MCP
28. Strong and oriented immobilization of single domain antibodies from crude bacterial lysates for high-throughput compatible cost-effective antibody array generation. (2010) Mol. Biosyst. 6, 2241-2248 Even-Desrumeaux K., Baty D., Chames P.
29. Damien Nelvoltris Conformational Nanobodies Reveal Tethered Epidermal Growth Factor Receptor Involved in EGFR/ErbB2 Predimers acsNANO 2015
30. Rozan C, Cornillon A, Petiard C, Chartier M, Behar G, Boix C, Kerfelec B, Robert B, Pelegrin A, Chames P, Teillaud J L and Baty D. Single-domain antibody-based and linker-free bispecific antibodies targeting FcgammaRIII induce potent antitumor activity without recruiting regulatory T cells. Mol Cancer Ther. 2013; 12(8):1481-1491.

1. An single domain antibody directed against natural killer group 2 member D (NKG2D) comprising:
a CDR1 having least 70% of identity with a sequence set forth as SEQ ID NO:1,
a CDR2 having at least 70% of identity with a sequence set forth as SEQ ID NO:2,
a CDR3 having at least 70% of identity with a sequence set forth as SEQ ID NO:3;
wherein said single domain antibody has a sequence with at least 95% identity to the sequence set forth as SEQ ID NO:4 or
a CDR1 having least 70% of identity with a sequence set forth as SEQ ID NO:5,
a CDR2 having at least 70% of identity with a sequence set forth as SEQ ID NO:6,
a CDR3 having at least 70% of identity with a sequence set forth as SEQ ID NO:7,
wherein, said isolated single domain antibody has a sequence with at least 95% identity to the sequence set forth as SEQ ID NO: 8.

2. The single domain antibody according to claim 1, wherein the single domain antibody is a “humanized” single domain antibody.

3. (canceled)

4. A polypeptide comprising at least one single domain antibody according to claim 1.

5. The polypeptide of claim 4 further comprising at least one other single domain antibody.

6. The polypeptide of claim 4, wherein the polypeptide is a bispecific polypeptide.

7. The polypeptide of claim 4 wherein the at least one single domain antibody is linked to an Fc portion.

8. The polypeptide of claim 4 which comprises:
a) i) a first fusion protein wherein the CL constant domain of an antibody is fused by its N-terminal end to the C-terminal end of the at least one single domain antibody and ii) a second fusion protein wherein the CH1 constant domain of an antibody is fused by its N-terminal end to the C-terminal end of a single domain antibody directed against an antigen different from NKG2D; or
b) a first fusion protein wherein the CH1 constant domain of an antibody is fused by its N-terminal end to the C-terminal end of a single domain antibody directed against a an activating trigger molecule on an effector cell and a second fusion protein wherein the CL constant domain of an antibody is fused by its N-terminal end to the C-terminal end of the at least one single domain antibody.

9. The polypeptide of claim 4 which comprises:
a) i) a first single domain antibody comprising a CDR1 having least 70% of identity with sequence set forth as SEQ ID NO:1, a CDR2 having at least 70% of identity with sequence set forth as SEQ ID NO:2 and a CDR3 having at least 70% of identity with sequence set forth as SEQ ID NO:3 and ii) a second single domain antibody against an epitope, antigen or polypeptide which is present on tumor cells;
b) i) a first single domain antibody comprising a CDR1 having least 70% of identity with sequence set forth as SEQ ID NO:1, a CDR2 having at least 70% of identity with sequence set forth as SEQ ID NO:2 and a CDR3 having at least 70% of identity with sequence set forth as SEQ ID NO:3 and ii) a second single domain antibody which is anti HER-2;
c) i) a first single domain antibody comprising a CDR1 having least 70% of identity with sequence set forth as SEQ ID NO:5, a CDR2 having at least 70% of identity with sequence set forth as SEQ ID NO:6 and a CDR3 having at least 70% of identity with sequence set forth as SEQ ID NO:7 and ii) a second single domain antibody against an epitope, antigen or polypeptide which is present on tumor cells; or
d) i) a first single domain antibody comprising a CDR1 having least 70% of identity with sequence set forth as SEQ ID NO:5, a CDR2 having at least 70% of identity with sequence set forth as SEQ ID NO:6 and a CDR3 having at least 70% of identity with sequence set forth as SEQ ID NO:7 and ii) a second single domain antibody which is anti HER-2.

10. The polypeptide of claim 4 wherein the two single domain antibodies are linked to each other directly or via a linker.

11. (canceled)

12. A nucleic acid encoding for a single domain antibody according to claim 1 or a polypeptide comprising the single domain antibody.

13. A vector which comprises the nucleic acid of claim 12.

14. A host cell which is transformed with the nucleic acid sequence of claim 12 or with a vector comprising the nucleic acid sequence.

15. A method for detecting NK cells in a biological sample obtained from a subject, comprising
contacting the biological sample with a single domain antibody of claim 1 or a polypeptide comprising the single domain antibody, and
detecting antibody-antigen complexes formed by the single domain antibody or the polypeptide comprising the single domain antibody and the NK cells.

16. A method for treating tumours, infectious diseases or autoimmune diseases in a subject in need thereof comprising administering to said subject a therapeutically effective amount of a single domain antibody according to claim 1 or a polypeptide comprising the single domain antibody.

17. A pharmaceutical composition comprising a single domain antibody according to claim 1 or a polypeptide comprising the single domain antibody.
